(19) United States
(12) Patent Application Publication
(10) Pub. No.: US 2022/0362210 A1
(43) Pub. Date: Nov. 17, 2022

(54) MINI-TABLETS IN CAPSULE DOSAGE FORM COMPRISING ATORVASTATIN

(71) Applicant: Luckwel Pharmaceuticals Inc., Cambridge, MA (US)

(72) Inventor: Kingrich Lee, Camrbidge, MA (US)

(21) Appl. No.: 17/732,113

(22) Filed: Apr. 28, 2022

Related U.S. Application Data
(60) Provisional application No. 63/181,062, filed on Apr. 28, 2021.

(51) Int. Cl. A61K 31/40; A61K 9/48

(52) U.S. Cl. CPC A61K 31/40; A61K 9/4858; A61K 9/485; A61K 9/4866; A61K 9/4875; A61K 9/4816

(57) ABSTRACT

A mini-tablets in capsule oral dosage form comprising a dose of atorvastatin. Also disclosed are methods of preparing the dosage form and methods of treating a condition, disease or disorder that is therapeutically responsive to atorvastatin.

### CROSS-REFERENCE TO RELATED APPLICATIONS

This application claims the benefit of and priority to U.S. Provisional Patent Application Ser. No. 63/181,062, entitled “MINI-TABLETS IN CAPSULE DOSAGE FORM COMPRISING ATORVASTATIN” and filed on Apr. 28, 2021, the disclosure of which is hereby incorporated by reference in its entirety for all purposes.

### TECHNICAL FIELD

The present subject matter relates to a mini-tablets in capsule dosage form of atorvastatin. Also described are methods of use of the dosage form for the treatment of diseases, disorders or conditions that are therapeutically responsive to atorvastatin. A process for preparing the mini-tablet dosage form is also provided.

### BACKGROUND

Atorvastatin is a drug for the treatment and prevention of cardiovascular and related diseases. The U.S. Food and Drug Administration (FDA) approved it in 1996. Solid dosage forms of atorvastatin, such as [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1), described in U.S. Pat. No. 5,273,995, are known (FDA Electronic Orange Book). Solid tablets are currently available under multiple trademarks, e.g., LIPITOR® (NDA N020702, UPJOHN MANUFACTURING IRELAND UNLTD., approval date Dec. 17, 1996; package insert available at https://dailymed.nlm.nih.gov/dailymed/drugInfo.cfm?setid=c6e131fe-e7df-4876-83f7-9156fc4e8228&audience=consumer. Tablets of various dosages are available including 10, 20, 40, or 80 mg of atorvastatin and the following excipients (inactive ingredients): anhydrous lactose, colloidal silicon dioxide, copovidone, croscarmellose sodium, magnesium stearate, mannitol, silicified microcrystalline cellulose, sodium bicarbonate, sodium carbonate anhydrous, sodium lauryl sulfate, lecithin, polyvinyl alcohol part hydrolyzed, talc, titanium dioxide, xanthan gum and iron oxide yellow.

Atorvastatin has the following structure:

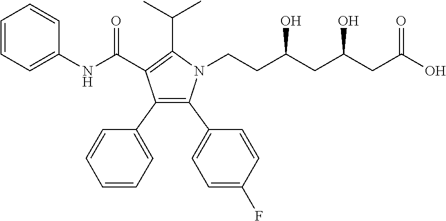 [SMILES: CC(C)c1c(C(=O)Nc2ccccc2)c(-c2ccccc2)c(-c2ccc(F)cc2)n1CC[C@@H](O)C[C@@H](O)CC(=O)O]

Atorvastatin is a white to off-white solid which is slightly soluble in water above pH 4. Atorvastatin has been found to be stable in a wide range of pharmaceutical formulations.

Atorvastatin is dosed at levels between 10 and 80 mg once daily and reaches maximum plasma concentrations within 1-2 hours. Atorvastatin has a bioavailability of just 14%, and has an unreliable Cₘₐₓ depending the patient population: geriatric population have 40% higher Cₘₐₓ and 30% higher AUC than young adults, women 20% higher Cₘₐₓ but 10% lower AUC than men, and 4-times higher in patients with hepatic insufficiency (e.g., chronic alcoholic liver disease). One potential solution to the unreliable dosing between patient populations is the use of mini-tablets, in which myriad doses and solid dosage forms may be inside a single orally available capsule.

It would be beneficial to provide additional dosage forms containing atorvastatin that exhibit superior and/or tailorable dosing for various patient populations; however, no such suitable dosage form containing atorvastatin has been disclosed in the art.

### BRIEF SUMMARY

In one aspect, the invention features a dosage form with a plurality of mini-tablet coated particles in a capsule, the particles having a core coated with an enteric coating; the core comprises atorvastatin; and the dosage form has about 2 mg to about 90 mg of Atorvastatin, such as about 20 mg, about 22.5 mg, about 40 mg, about 45 mg, about 80 mg and about 90 mg. In one embodiment, the dosage form has 22.5 mg Atorvastatin. In another embodiment, the dosage form has 45 mg Atorvastatin. In a further embodiment, the dosage form has 90 mg Atorvastatin.

In one aspect, the invention features a dosage form containing a plurality of coated particles, the particles having a core coated with an enteric coating, and the core containing atorvastatin.

In some embodiments, the core also contains at least one disintegrant, at least one glidant, at least one polymeric binder, and at least one lubricant.

In some embodiments, the at least one disintegrant is croscarmellose sodium, the at least one glidant is colloidal silicon dioxide, the at least one polymeric binder is microcrystalline cellulose, the at least one lubricant is magnesium stearate, and the enteric polymer is hydroxypropylmethylcellulose phthalate.

In some embodiments, the core also contains at least one plasticizer. In one embodiment the at least one plasticizer is hydrogenated castor oil. In another embodiment, the at least one plasticizer is triethyl citrate.

In some embodiments, the dosage form also has second active pharmaceutical ingredient.

In some embodiments, the plurality of coated particles are between 1 and 5 mm in diameter.

In some embodiments, the dosage form is a capsule made of hydroxypropylmethyl cellulose, filled with the plurality of coated particles. In one embodiment, the plurality of coated particles are non-uniform in diameter.

In another aspect, the invention features a method of treating or preventing a disease or disorder in a human, such as a cardiovascular disease or disorder, by lowering the levels of cholesterol and triglycerides in the blood by administering the dosage form of described herein to a subject in need thereof. In some embodiments, the cardiovascular disease or disorder is congestive heart failure, or coronary heart disease.

### DETAILED DESCRIPTION

### I. DEFINITIONS

Various aspects now will be described more fully hereinafter. Such aspects may, however, be embodied in many different forms and should not be construed as limited to the embodiments set forth herein; rather, these embodiments are provided so that this disclosure will be thorough and complete, and will fully convey its scope to those skilled in the art.

Where a range of values is provided, it is intended that each intervening value between the upper and lower limit of that range and any other stated or intervening value in that stated range is encompassed within the disclosure. For example, if a range of 1 ml to 8 ml is stated, it is intended that 2 ml, 3 ml, 4 ml, 5 ml, 6 ml, and 7 ml are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to an “excipient” includes a single excipient as well as two or more of the same or different excipients, and the like.

“About” or “approximately” as used herein means within an acceptable error range for the particular value as determined by one of ordinary skill in the art, which will depend in part on how the value is measured or determined, (i.e., the limitations of the measurement system). For example, “about” can mean within 1 or more than 1 standard deviations, per practice in the art. Where particular values are described in the application and claims, unless otherwise stated, the term “about” means within an acceptable error range for the particular value. In some embodiments, “about” means that the item, parameter or term so qualified encompasses a range of plus or minus ten percent above and/or below the value of the stated item, parameter or term.

“Administration”, or “to administer” means the step of giving (i.e. administering) a pharmaceutical composition to a subject, or alternatively a subject receiving a pharmaceutical composition. The pharmaceutical compositions disclosed herein can be locally administered by various methods. For example, intramuscular, intradermal, subcutaneous administration, intrathecal administration, intraperitoneal administration, topical (transdermal), instillation, and implantation (for example, of a slow-release device such as polymeric implant or miniosmotic pump) can all be appropriate routes of administration.

“Alleviating” means a reduction in the occurrence of a pain, of a headache, or of any symptom or cause of a condition or disorder. Thus, alleviating includes some reduction, significant reduction, near total reduction, and total reduction.

“Therapeutically effective amount” as applied to the biologically active ingredient means that amount of the active ingredient which is generally sufficient to effect a desired change in the subject. For example, where the desired effect is a reduction in an autoimmune disorder symptom, an effective amount of the active ingredient is that amount which causes at least a substantial reduction of the autoimmune disorder symptom, and without resulting in significant toxicity.

“Subject” or “patient” means a human or non-human subject receiving medical or veterinary care. Accordingly, the method as disclosed herein can be used in treating any animal, such as, for example, mammals, or the like.

“Treating” means to alleviate (or to eliminate) at least one symptom of a condition or disorder, such as, for example, cough, fever, shortness of breath, or the like, either temporarily or permanently.

### II. ATORVASTATIN DOSAGE FORM

As used herein and unless otherwise specified, the term atorvastatin refers to the drug in underivatized as R-(R*,R*)]-2-(4-fluorophenyl)-β,δ-dihydroxy-5-(1-methylethyl)-3-phenyl-4-[(phenylamino)carbonyl]-1H-pyrrole-1-heptanoic acid, calcium salt (2:1) described in U.S. Pat. No. 5,273,995 or derivatized form. Atorvastatin can be present in crystalline or amorphous form. All polymorphs of crystalline atorvastatin and mixtures thereof can be used.

Provided is a pharmaceutical composition suitable for administration to a subject, the composition comprising atorvastatin. In some embodiments, atorvastatin represents between 1% and 50% of the composition by weight. In some embodiments, atorvastatin represents between 1% and 10% of the composition by weight, e.g., between 1% and 2%, between 2% and 3%, between 3% and 4%, between 4% and 5%, between 5% and 6%, between 6% and 7%, between 7% and 8%, between 8% and 9%, or between 9% and 10% by weight.

In some embodiments, the dosage form is self-preserved and does not require the addition of a preservative, even though a preservative can be included if desired. Accordingly, some embodiments provide a preservative-free mini-tablet dosage of atorvastatin.

Some embodiments provide a mini-tablet dosage form comprising atorvastatin, diluent, disintegrant, and one or more other excipients, wherein the dosage form is stable to oxidative degradation of atorvastatin when stored at 21° C. for six months at 75% relative humidity (RH).

The drug release profile of the mini-tablet dosage form may be sustained, extended, or delayed release, or a suitable combination thereof. A single capsule of such mini-tablets may contain individual doses among which some are sustained, extended, or delayed release, or a combination thereof. The sustained, extended, or delayed release of the atorvastatin mini-tablets provide an optimal plasma drug concentration.

In some aspects, a mini-tablet dosage form and administration thereof for the treatment of diseases, conditions or disorders that are therapeutically responsive to atorvastatin, such as cardiovascular and related diseases are provided.

In some embodiments, the dosage form contains about 20 mg to about 90 mg of Atorvastatin, such as about 20 mg, about 22.5 mg, about 40 mg, about 45 mg, about 80 mg or about 90 mg.

### III. EXCIPIENTS

The compositions or oral dosage forms of the present invention can also further comprise one or more stabilizers which enhance or improve the stability of the compositions or oral dosage forms of the present invention. Non-limiting examples of suitable stabilizers include proline, trehalose, dextran, maltose, Sucrose, mannitol, polyols, silica gel, aminoguanidine, pyridoxamine, anhydrous metal salts, such as sodium hydrogen carbonate magnesium oxide, calcium oxide, aluminum oxide and mixtures thereof. The one or more stabilizers can have a moisture content of about 3% or less and/or a water activity of 0.6 or less.

Non-limiting examples of suitable binders include starches, sugars (e.g. lactose), sugar alcohols (e.g. xylitol, sorbitol, maltitol), cellulose (e.g. microcrystalline cellulose), modified celluloses (e.g., hydroxypropylcellulose, carboxymethylcellulose sodium), alginic acid, polyvinyl pyrrolidone (povidone), and mixtures thereof.

Non-limiting examples of suitable disintegrants include dibasic calcium phosphate, dibasic calcium phosphate dihydrate, tribasic calcium phosphate, alginic acid, hydroxypropylcellulose, carboxymethylcellulose calcium, carboxymethylcellulose sodium, cross-linked carboxymethylcellulose sodium, swellable ion exchange resins, alginates, formaldehyde casein, cellulose, croscarmellose sodium, crospovidone (e.g., cross-linked polyvinyl pyrrolidone), microcrystalline cellulose, sodium carboxymethyl starch, sodium starch glycolate, starches (corn starch, rice starch), and mixtures thereof.

Non limiting examples of suitable lubricants include calcium stearate, magnesium stearate, sodium stearyl fumarate, stearic acid, zinc stearate, talc, waxes, SteroteXCR, Stearowet(R), and mixtures thereof.

Non-limiting examples of suitable glidants include colloidal silicon dioxide, talc, and mixtures thereof.

Non-limiting examples of suitable diluents include mannitol, sucrose, anhydrous dibasic calcium phosphate, anhydrous dibasic calcium phosphate dihydrate, tribasic calcium phosphate, cellulose, lactose, magnesium carbonate, microcrystalline cellulose, and mixtures thereof.

Non limiting examples of suitable stabilizers include trehalose, proline, dextran, maltose, sucrose, mannitol, polyols, silica gel, aminoguanidine, pyridoxamine, and mixtures thereof. In one embodiment, the disintegrant is crospovidone (e.g., POLYPLASDONE XL, POLYPLASDONE XL-10). In another embodiment, the disintegrant is croscarmellose sodium (e.g., AC-DI-SOL). In another embodiment, the disintegrant is sodium starch glycolate (e.g., EXPLOTAB, EXPLOTAB CV). In another embodiment, the compositions or oral dosage forms of the present invention can comprise a combination of disintegrants such as microcrystalline cellulose and sodium starch glycolate or croscarmellose sodium and crospovidone. The amount of disintegrant can be in the range of about any of about 0.1-30%, 1%-30%, 1%-25%, 1%-20%, 1%-15%, 1%-10%, 1%-5%. 5%-10%, 5%-15%, 5%-20%, 5%-25%, or 5%-30%. In one embodiment, the amount of disintegrant is about 2%-4%, or about 2%-3%, or about 2.5%.

Non-limiting examples of suitable diluents include micro crystalline cellulose, starch, calcium phosphate, lactose, sucrose, mannitol, sorbitol, and combinations thereof. In one embodiment, the diluent is microcrystalline cellulose (e.g. Avicel). In another embodiment, the diluent is starch. In another embodiment, the diluent is lactose (e.g., Pharmatol). In another embodiment, the compositions or oral dosage forms of the present invention can comprise a combination of diluents such as microcrystalline cellulose, starch and lactose. The amount of diluent can be in the range of about any of about 0.1-99%, 1%-30%, 1%-25%, 1%-20%, 1%-15%, 1%-10%, 1%-5%. 5%-10%, 5%-15%, 5%-20%, 5%-25%, or 5%-30%. In one embodiment, the amount of diluent is about 2%-5%, 3%-5%, or about 4%. One or more of the excipients of the compositions or oral dosage forms of the present invention can function as a desiccant to further stabilized the composition. Suitable excipients useful as desiccants include any pharmaceutically acceptable excipient that binds water tightly, or reduces the water activity of a composition. For example, the composition of the present invention can include about 1-4% silica gel, or about 2.5% silica gel. The compositions of the present invention can be prepared in any suitable oral dosage form.

Non-limiting examples of suitable dosage forms include tablets, capsules or sachets. Since atorvastatin may need to be protected during digestion, it may be desirable that the present invention be provided as a controlled or delayed release formulation. Such controlled or delayed release formulations can include tablets or particles coated with an enteric coating which serves to protect the sensitive API through part of digestion. Alternatively, the controlled release formulations can include capsules filled with the API or oral dosage forms of the present invention, whereby the capsule protects the contents against, e.g., gastric inactivation. The term “particles” as used herein includes fine powders (having particle diameters in the range of about 1 um) up to pellets having a diameter of about 5 mm. The API can also be formed into particles coated with a coating, wherein the coating comprises an enteric polymer. The term “enteric polymer” means a polymer that protects the API from gastric contents, for example a polymer that is stable at acidic pH, but can break down rapidly at higher pH or a polymer whose rate of hydration or erosion is slow enough to ensure that contact of gastric contents with the API is relatively minor while it is in the stomach, as opposed to other portions of gastro-intestinal tract. Non limiting examples of enteric polymers include those known in the art, such as modified or unmodified natural polymers such as cellulose acetate phthalate, hydroxypropylmethylcellulose phthalate, hydroxypropylmethylcellulose acetate succinate, and shellac, or synthetic polymers such as acrylic polymers or copolymers methacrylic acid polymers and copolymers, methylmethacrylate copolymers, and methacrylic acid/methylmethacrylate copolymers. The enteric polymer coating can be a synthetic polymer, optionally including an inorganic material, such as an alkalinizing agent. The resulting coated particles provide delayed release beads comprising a core which comprises the API and an enteric coating encapsulating the core. The coated API particles can then be formulated into tablets or capsules.

The enteric polymer and the at least one inorganic material impart enteric properties to the coating of the present invention. That is, when used as a medication, the coating will act as a barrier protecting the medication from the acidic environment of the stomach and substantially prevent the release of the medication during a part of the digestive process. The inorganic material can include, for example, an alkalinizing agent. Non-limiting examples of alkalinizing agents include silicon dioxide, sodium salts, calcium salts, magnesium salts, aluminum salts, aluminum hydroxides, calcium hydroxides magnesium hydroxides, talc, and combinations thereof. In one embodiment, the alkalinizing agent is talc. Depending on the intended use of the composition, the ratio of the enteric polymer and the at least one inorganic material may be in a range of from about 10:1 to about 1:60 by weight, (e.g., about 10:1 to about 5:1, about 5:1 to about 1:1, about 1:1 to about 1:5, about 1:5 to about 1:10, about 1:10 to about 1:20, about 1:20 to about 1:30, about 1:30 to about 1:45, or about 1:45 to about 1:60). In one embodiment, the compositions or oral dosage forms of the present invention comprise API coated with an enteric coating comprising an enteric polymer and an inorganic material such as talc. In a particular embodiment, the inorganic material of the enteric coating comprises about 1-10% by weight of the weight of the total weight of the particles. In another embodiment the inorganic material comprises about 3, about 5, about 7, or about 10% by weight of the particles. In still other embodiments, the inorganic material is an alkalinizing agent and comprises about 20-60% of the dry coating weight. In still other embodiments, the alkalinizing agent is about 25%, about 30%, about 35%, about 40%, about 45%, about 50%, or about 55% of the dry coating weight (inclusive of all ranges, subranges, and values therebetween). In a particular embodiment, the alkalinizing agent is talc. In still another particular embodiment, the dry coating of the particles comprises about 35% talc. In another embodiment of the present invention, the coating further comprises a plasticizer. Examples of suitable plasticizers include, but are not limited to triacetin, tributyl citrate, tri-ethyl citrate, acetyl tri-n-butyl citrate, diethyl phthalate, dibutyl sebacate, polyethylene glycol, polypropylene glycol, castor oil, acetylated mono-glyceride, acetylated di-glyceride, and mixtures thereof. The dosage forms of the present invention can be capsules containing the composition of the present invention (e.g., controlled-release particles of the API composition, coated with an enteric polymer and an alkalinizing agent). The capsules themselves can be comprised of any conventional biodegradable material known in the art, for example, gelatin, polysaccharides such as pullulan, or modified cellulosic materials such as hydroxypropyl methylcellulose. In order to improve the stability of the API, the capsule can be dried prior to filling, or a capsule comprised of a low moisture content material can be selected. In one embodiment of the dosage form of the present invention, the capsule is comprised of hydroxypropylmethylcellulose. In another embodiment of the dosage form of the present invention, the capsule is comprised of hydroxypropylmethylcellulose having a water con tent of about 6% or less, for example about any of 4% or less, 2% or less, or 2-6%, or 4-6%. In another embodiment, the capsule is comprised of hydroxypropylmethylcellulose having a water content of less than about 2%. The dosage form can also comprise coated particles, each of which has nominally the same composition, or it can comprise mixtures of different kinds of coated particles. Any suitable combination of coated particles of different compositions can be used to provide the desired therapeutic effect. Any suitable combination of coating compositions for different coated particles can be used to provide the desired type of controlled release or therapeutic effect.

The core of the coated particles can have any suitable particle size or shape. For example, the coated particles can be in the form of mini-tablets which have a nominal particle diameter in the range of about 1-5 mm.

In one embodiment, the API particles can comprise about 60-90% of atorvastatin, about 1-4% of at least one disintegrant, about 2-6% of at least one polymeric binder or diluent, and optionally about 0.5-1.0% of at least one plasticizer, optionally about 0.2-0.6% of at least one glidant, and optionally about 0.2-0.6% of at least one lubricant. For example, the API particles can comprise about 60-90% atorvastatin, about 1-4% of croscarmellose sodium, about 0.5-1.0% of hydrogenated castor oil, about 0.2-0.6% of colloidal silicon dioxide, about 2-6% of microcrystalline cellulose, and about 0.2-0.6% of magnesium stearate. The coating can comprise at least one enteric polymer, about 4-10% of at least one alkalinizing agent (based on the total weight of the particles), and optionally at least one plasticizer. In one embodiment, the coating can comprise about 10-20% of at least one enteric polymer, about 4-10% of a least one alkalinizing agent, and about 1-2% of a least one plasticizer (based on the total weight of the particles). For example, the coating can comprise about 10-20% of hydroxypropylmethylcellulose phthalate, about 4-10% of talc, and about 1-2% of triethylcitrate (based on the total weight of the particles). The plurality of coated API particles can then be formed into a tablet, or filled into a capsule. In one embodiment, the capsule comprises hydroxypropylmethyl cellulose. The compositions of the present invention, and dosage forms comprising the compositions of the present invention, have improved absorption profiles compared to conventional atorvastatin compositions and dosage forms.

The compositions or dosage forms (e.g., tablets or capsules) of the present invention can be stored in any suitable package. For example, the package can be a glass or plastic jar with a threaded or press-fit closure. Alternatively, the compositions or dosage forms of the present invention can be packaged as a unit dosage form in “blister packs”. The package may also be made of moisture-proof packaging. Non limiting examples of suitable moisture-proof packaging include glass jars, plastic jars incorporating moisture barrier resins or coatings, aluminized plastic (e.g., Mylar) packaging, etc. The term “moisture-proof” refers to a package which has a permeability to water of less than about 0.5 mg water per cm of container volume per year. Containers (e.g., bottles) can be closed with any suitable closure, especially closures which minimize the ingress of moisture during storage.

Packages containing the compositions or dosage forms of the present invention can also contain a desiccant (i.e., a substance which absorbs, reacts with, or adsorbs water) capable of reducing the humidity inside the package, for example a desiccant capsule, capable of scavenging moisture from the atmosphere sealed inside the package. Non limiting examples of suitable desiccants which can be placed inside such packages include Zeolites (e.g., molecular sieves such as 4A molecular sieves), clay (e.g., montmorillonite clay), silica gel, activated carbon, or combinations thereof. In one embodiment, the desiccant comprises molecular sieves.

For patients identified as having low GI pH levels (e.g., GI pH levels<about 4), improved results may be obtained by administering the compositions or dosage forms of the present invention together with proton pump inhibitors, antacids, and other drugs which increase the pH of the GI tract. For example, the compositions or dosage forms of the present invention can be administered separately from the proton pump inhibitors, antacid, or other drugs (either prior to, concurrently with, or after administration of the proton pump inhibitor, antacid, etc.).

In yet another embodiment, the present invention provides a method of treating or preventing a cardiovascular or related disorder comprising administering a composition of the present invention to a mammal in need thereof. In one embodiment, the mammal is a human.

In some embodiments, the cardiovascular disorder is congestive heart failure or coronary heart disease. In yet another embodiment, the cardiovascular or related disorder stems from or is promoted by, hyperlipidemia or hypercholesterolemia.

The amount of the composition or dosage form of the present invention administered daily to mammals (e.g., humans) depends upon the intended result. The skilled physician will be capable of prescribing the required dose based on his diagnosis of the condition to be treated.

For example, for the treatment of congestive heart failure in humans the starting dose should be 10 to 20 mg, with the total dose not exceeding 80 mg in accordance with the recommendations of the US FDA.

### IV. EXAMPLES

The following examples are illustrative in nature and are in no way intended to be limiting.

### Example 1

Atorvastatin MT (minitabs) is a blend of Atorvastatin raw material and excipients (e.g., croscarmellose sodium, hydrogenated castor oil, colloidal silicon dioxide, microcrystalline cellulose, and magnesium stearate; Table 1) tableted using round 2mm diameter beveled punches.

TABLE 1
Content (mg/capsule) for each Dosage Strength

| | Composition | Composition | Composition |
|---|---|---|---|
| | 1 | 2 | 3 |
| Component | (MT) | (MT) | (MT) |
| Mini-tablets | | | |
| Atorvastatin | 22.5 | 45 | 90 |
| Croscarmellose Sodium | 3.6 | 5.5 | 7.3 |
| Hydrogenated Castor Oil | 1.2 | 1.8 | 2.4 |
| Colloidal Silicon Dioxide | 0.6 | 0.9 | 1.2 |
| Microcrystalline Cellulose | 6.1 | 9.1 | 12.1 |
| Magnesium Stearate | 0.6 | 0.9 | 1.2 |
| Coating | | | |
| Hypromellose Phthalate | 18.9 | 28.4 | 37.8 |
| Talc | 9.5 | 14.2 | 18.9 |
| Triethyl Citrate | 1.92 | 2.8 | 3.8 |
| Acetone | Trace | Trace | Trace |
| Capsule | | | |
| Carrageenan | 0.2 | 0.3 | 0.3 |
| Potassium Chloride | 0.3 | 0.4 | 0.4 |
| Titanium Dioxide | 3.5 | 5.1 | 5.2 |
| Hypromellose | 52.9 | 79.4 | 79.2 |
| Carnauba Wax | Trace | Trace | Trace |
| Water | 0.60 | 0.9 | 0.90 |
| Yellow Iron Oxide | 0.1 | — | 0.2 |
| Red Iron Oxide | — | 0.3 | — |
| FDC Blue 2 | — | — | 0.1 |

The physical characteristics of Atorvastatin Mini-tablets before coating are shown in

Table 2.

TABLE 2

| | | |
|---|---|---|
| Diameter | 2.0 | mm |
| Weight (of 10 Mini-tablets) | 0.074-0.086 | g |
| Thickness (mean value of 10 Mini-tabiets) | 2.2 ± 0.2 | mm |
| Hardness | 0.5-2.0 | Kp |

| | |
|---|---|
| Friability (20 g of Mini-tabiets-30 min at 2.5 rpm) | 0.0-2.5% |

What is claimed is:

1. A dosage form comprising a plurality of mini-tablet coated particles in a capsule, the particles comprising a core coated with an enteric coating; wherein the core comprises atorvastatin, and wherein the dosage form comprises about 2 mg to about 90 mg of Atorvastatin, such as about 20 mg, about 22.5 mg, about 40 mg, about 45 mg, about 80 mg and about 90 mg.

2. The method according to claim 1, wherein the dosage form comprises 22.5 mg Atorvastatin.

3. The method according to claim 1, wherein the dosage form comprises 45 mg Atorvastatin.

4. The method according to claim 1, wherein the dosage form comprises 90 mg Atorvastatin.

5. The dosage form of claim 1, wherein the core further comprises at least one disintegrant; at least one glidant; at least one polymeric binder; and at least one lubricant.

6. The dosage form of claim 1 or claim 2, wherein the at least one disintegrant is croscarmellose sodium, the at least one glidant is colloidal silicon dioxide, the at least one polymeric binder is microcrystalline cellulose, the at least one lubricant is magnesium stearate, and the enteric polymer is hydroxypropylmethylcellulose phthalate.

7. The dosage form of any one of claims 1 to 6, wherein the core further comprises at least one plasticizer.

8. The dosage form of claim 7, wherein the at least one plasticizer is hydrogenated castor oil.

9. The dosage form of claim 7, wherein the at least one plasticizer is triethyl citrate.

10. The dosage form of any one of claims 1 to 9, further comprising a second active pharmaceutical ingredient.

11. The dosage form of any one of claims 1 to 10, wherein the plurality of coated particles are between 1 and 5 mm in diameter.

12. The dosage form of any one of claims 1 to 11, wherein the dosage form is a capsule comprised of hydroxypropylmethyl cellulose, filled with the plurality of coated particles.

13. The dosage form of any one of claims 1 to 12, wherein the plurality of coated particles are non-uniform in diameter.

14. A method of treating or preventing a disease or disorder in a human, wherein the disease or disorder is a cardiovascular disease or disorder, by lowering levels of cholesterol and triglycerides in the blood, the method comprising administering the dosage form of any one of claims 1-13 to a subject in need thereof.

15. The method of claim 14, wherein the cardiovascular disease or disorder is congestive heart failure, or coronary heart disease.
